(19) United States
(12) Patent Application Publication
(10) Pub. No.: US 2023/0211027 A1
(43) Pub. Date: Jul. 6, 2023

(54) X-RAY IRRADIATION REAL TIME DOSE MEASUREMENT/MONITORING

(71) Applicant: RexSter Technologies, Inc., Acton, MA (US)

(72) Inventors: Stephen E. Korbly, Acton, MA (US); Jagadishwar R. Sirigiri, Natick, MA (US)

(21) Appl. No.: 18/146,668

(22) Filed: Dec. 27, 2022

Related U.S. Application Data
(60) Provisional application No. 63/296,686, filed on Jan. 5, 2022.

(51) Int. Cl. A61L 2/08; A61L 2/24

(52) U.S. Cl. CPC A61L 2/082; A61L 2/24; A61L 2202/122; A61L 2202/123; A61L 2202/11; A61L 2202/14

(57) ABSTRACT

A product sterilization system includes a vault, a source of X-rays in the vault producing a field of X-rays in a treatment zone, a conveyance delivering products to the treatment zone for irradiation by the field of X-rays, a first X-ray detection subsystem in the treatment zone between the products and the source of X-rays, a second X-ray detection subsystem in the treatment zone behind the products, and a controller subsystem responsive to the first X-ray detection subsystem and the second X-ray detection subsystem and configured to determine an X-ray dose absorbed by the products.

### RELATED APPLICATIONS

This application claims benefit of and priority to U.S. Provisional Application Ser. No. 63/296,686 filed Jan. 5, 2022, under 35 U.S.C. §§ 119, 120, 363, 365, and 37 C.F.R. § 1.55 and § 1.78, which is incorporated herein by this reference.

### FIELD OF THE INVENTION

This subject of the disclosure relates to the field of sterilization and irradiation of products or materials via X-rays.

### BACKGROUND OF THE INVENTION

Recent shortages of Personal Protective Equipment (PPE) during the COVID pandemic have highlighted the importance of sterilization of medical devices. Limitations of existing methods in the infrastructure of the medical device sterilization market due to environmental safety, nuclear proliferation risks and supply-demand challenges are causing delays, cost increases, and supply chain interruptions.

Sterilization is often a critical step in the manufacturing process of various health care and medical products. Historically, ethylene oxide (EtO) and radioactive Cobalt-60 gamma radiation have accounted for a large percentage of the global sterilization services market. Both alternatives entail safety, environmental, supply chain, quality, and cost risk that can render their continued use and growth in the market untenable. EtO is a toxic explosive gas that significantly contributes to potential elevated cancer risks and environmental harm. The manufacturing of Cobalt-60 generates nuclear waste. Moreover, the non-proliferation of Cobalt-60 and scale back in nuclear facilities has limited the available supply of Cobalt-60. These issues with both sterilization methods have recently been highlighted by U.S. government agencies such as the Food and Drug

Administration and the Department of Energy and recommended for replacement. In fact, supply chain interruptions to several types of medical devices have already occurred.

Sterilization by other kinds of radiation including, but not limited to, electron beam, X-ray, ultraviolet, infrared, ion beam, and laser sterilization is also known. See, for example, U.S. Pat. Nos. 4,652,763; 7,187,752; 2005/0135965; 7,780,920; 6,485,979; WO2007/127321; and U.S. Pat. No. 9,142,383 all incorporated herein by this reference.

In general, the sterilization of medical products depends on the ability of the process to kill pathogenic microorganisms. The application of radiation to sterile medical products is widely used throughout the world and is recognized as a safe, effective form of sterilization. The FDA requires that all processes used to produce medical devices be validated. See for example ISO 11137. The goal of validation is to determine the minimum exposure dose that can be used to meet a desired sterility assurance level and allow for dosimetric release—a determination that a product is sterile based on physical or radiation process data rather than actual sterility testing. Once a minimum dose has been set, the quality of sterilization can be determined by the dose of radiation applied to the kill zone. The dose is determined by factors such as beam current, electron energy, and beam scanning parameters of the X-ray source.

Sterilization of products via gamma radiation typically involves loading products into totes and introducing a plurality of totes either on a continuous conveyor or in bulk into a radiation chamber. Within the chamber the product stacks pass by a radioactive source until the desired radiation dosage is received by the product and the totes are removed from the chamber. The dose in the kill zone may be estimated by securing dosimeters to areas near the kill zone and correlating the dosage reading with known actual kill rates for the real dosage readings. Typically, dosimeters are applied to the kill zone in preliminary production runs. Alternatively, dosimeters can be applied to the outer packaging components throughout the production run and read after processing. Such methods are thus inconvenient and expensive.

### BRIEF SUMMARY OF THE INVENTION

One problem in the radiation processing of products is that the effectiveness of radiation processing is sensitive to variations in product density and geometry, product source geometry, and source intensity. The use of dosimeters on each product requires a person to place the dosimeter on the product, remove the dosimeter and read the dosimeter to verify a sufficient dose to the product. This approach requires additional manual or automatic mechanical interventions which reduces the throughput of the overall system. Furthermore, the accuracy of the dose is dependent on a strict a priori compliance of the product density profile which is subject to variations during packing and shipment.

The present solution provides a method for use in the validation of X-ray sterilization systems. The solution provides a method that cost effectively and reliably provide for the routine dosimetric monitoring of the sterilization process for X-ray sterilization systems. The dosimetry is in line and in real time and can be automated and correlated to a specific product. Furthermore, the proposed system and process can utilize measurements to determine the density profile of the product on the fly allowing for adjustment of the X-ray radiation profile and time of exposure to account for the measured density profile of the product.

The new process can guarantee meeting both the minimum dose requirements and avoiding any maximum exposure limit which can be important for most medical devices. Some materials, for example Teflon®, and polypropylene can degrade if too high an X-ray dose is used.

The use of real time dose monitors/detectors to monitor the dose provided to a product increases the efficiency both in time and cost for the sterilization process. X-ray sterilization techniques with a higher quality, higher throughput, lower cost, environmentally friendly, sterilization service will help secure the supply chain for the rapidly growing medical device market.

The dose of the output of the X-ray target is preferably measured via a dose monitoring sensor/device. The dose is monitored in real time and correlated with the product or material being sterilized. The transmitted dose can also be measured before and after the X-rays traverse through the product. The ratio of these two measurements provides information about how much dose was absorbed in the product. A segmented sensor can provide additional information about the specific material being irradiated.

This new method provides for real time measurement of the dose to the product which can enable parametric release.

Featured is a product sterilization system comprising a vault, a source of X-rays in the vault producing a field of X-rays in a treatment zone, and a conveyance delivering products to the treatment zone for irradiation by the field of X-rays. There is a first X-ray detection subsystem in the treatment zone between the products and the source of X-rays and a second X-ray detection subsystem in the treatment zone behind the products. A controller subsystem is responsive to the first X-ray detection subsystem and the second X-ray detection subsystem and is configured to determine an X-ray dose absorbed by the products.

The source of X-rays may include an electron accelerator and a target producing X-rays in response to electrons emitted by the electron accelerator. The conveyance may include a vault entrance conveyor, a vault exit conveyor for palletized products, and a return conveyor between the entrance conveyor and the exit conveyor configured to return products to the treatment zone. In that example, the controller subsystem is preferably further configured to direct the products from the exit conveyor to the return conveyor when the dose absorbed by the products is below a specified dose.

The first X-ray detection subsystem preferably includes one or more low attenuation detectors between the product and the source of X-rays. In one example, detectors are ion chamber detectors.

Also featured is a product sterilization method comprising conveying products to a treatment zone irradiated by a field of X-rays, measuring the intensity of the X-rays in the treatment zone before the X-rays irradiate the products, measuring the intensity of the X-rays in the treatment zone behind the products after the X-rays pass through the products, and automatically determining an X-ray dose absorbed by the products based on the measured intensity of the X-rays in the treatment zone before the X-rays irradiate the product and the measured intensity of the X-rays in the treatment zone behind the products after the X-rays pass through the products. One preferred method includes automatically calculating when the dose absorbed by the products is below a specified dose and if so automatically returning the products via a conveyance to the treatment zone.

One product sterilization method includes conveying products to a treatment zone irradiated by a field of X-rays, where the X-ray flux before and after traversing through the products is mapped, calibrated, and stored in a database and with such information either directly or scaled applied to the same process in a different facility with an X-ray source of either the same or different power.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic view showing an example of a product sterilization system;

FIG. 2 is a schematic view showing medical devices boxed and palletized on a conveyor entering the sterilization treatment zone for irradiation by a field of X-rays in the zone;

FIGS. 3-4 are block diagrams showing the primary components associated with a preferred product sterilization system;

FIGS. 5-7 are a schematic view showing the primary steps associated with an example of a product sterilization method and also showing the programming algorithm associated with the controller or processor of FIG. 3;

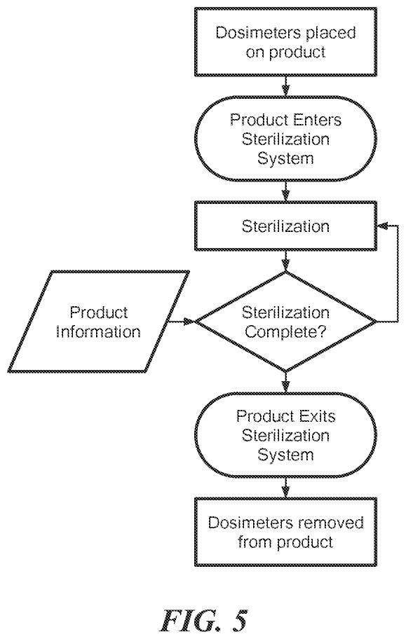

FIG. 8 is a graph of attenuation versus product length of a low density, homogenous product subject to single sided irradiation;

FIG. 9 shows an example of a low density, homogenous product with dual side irradiation;

FIG. 10 shows an example of low-density homogenous product total irradiation; and

FIG. 11 is a graph showing an example of a high density, homogenous product.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

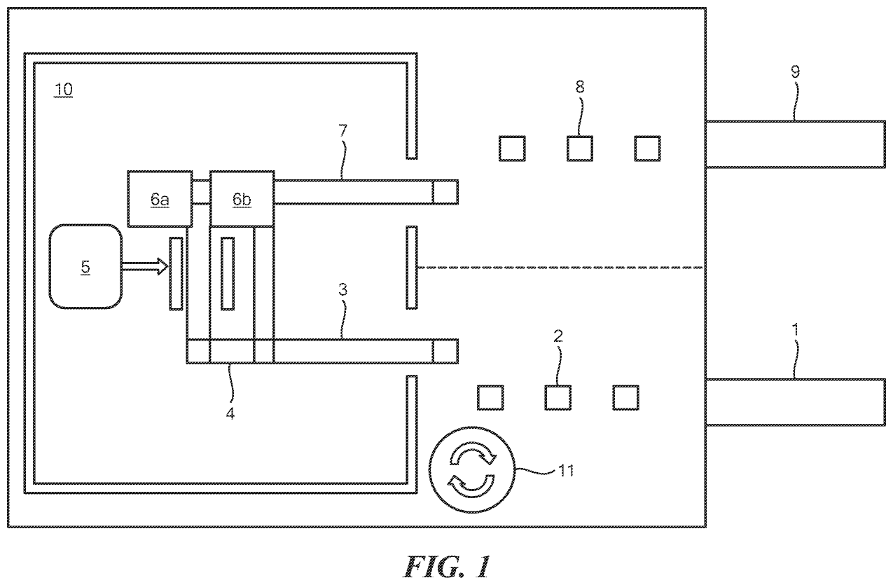

FIG. 1 shows an embodiment of an X-ray sterilization facility in one example. A truck arrives at facility 12 and offloads unsterilized product typically packaged on pallets 18 onto the infeed of vault entrance conveyor 14. The pallets are loaded onto conveyor 14 at the entrance to shielding vault 16. Pallets 18 pass in front of an X-ray source 20. In this embodiment, one or more detectors 22a are placed in front of the pallet and one or more detectors 22b are placed behind the pallet to measure the incoming and outgoing dose. The process control subsystem 30 monitors the sterilization process and when the process is complete the pallets exit the vault on vault exit conveyor 32. The sterilized products may undergo a quality check and then the pallets are loaded onto another truck at the outfeed of vault exit conveyor 32.

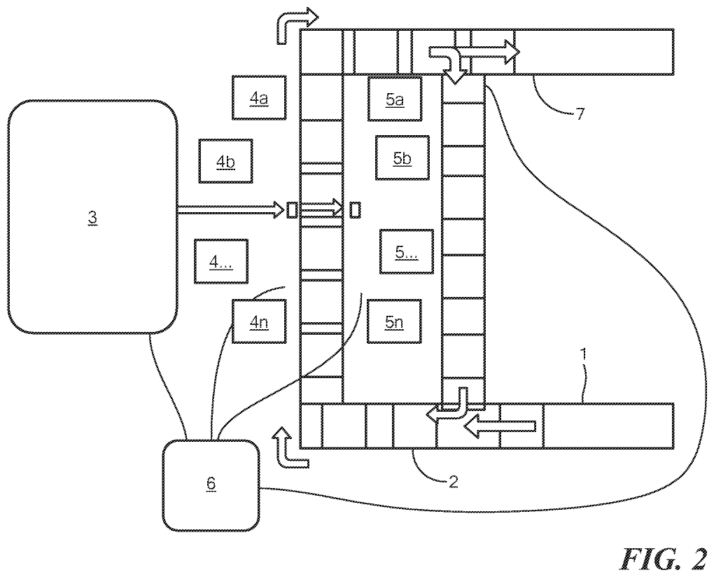

As shown in FIG. 2, the X-ray source includes, for example, an electron accelerator producing a beam of electrons which impinge on a suitable target to produce an X-ray field in treatment zone 40. Accordingly, collimation may not be required. Conveyance 24 delivers pallet 18 to zone 40 for irradiation by X-rays. The pallet may include boxes 43 each of which includes medical devices, for example, nasal swabs, implants, and the like. In some embodiments, a complete pallet includes boxes all filled with the same type of medical device but in other examples a pallet could include different boxes with different types of medical devices packaged therein.

A first X-ray detector subsystem in the treatment zone is typically fixed in place between pallet 18 and the source of X-rays. In one example, the first X-ray detection subsystem includes one or more low attenuation detectors such as an ion chamber detector array 22a. Scintillation type detectors may also be used. A second X-ray detector detection subsystem 22b is typically fixed in place behind pallet 18 in zone 40. Again, one or more low attenuation detectors such as an ion chamber detection array and/or scintillation type detectors may be used.

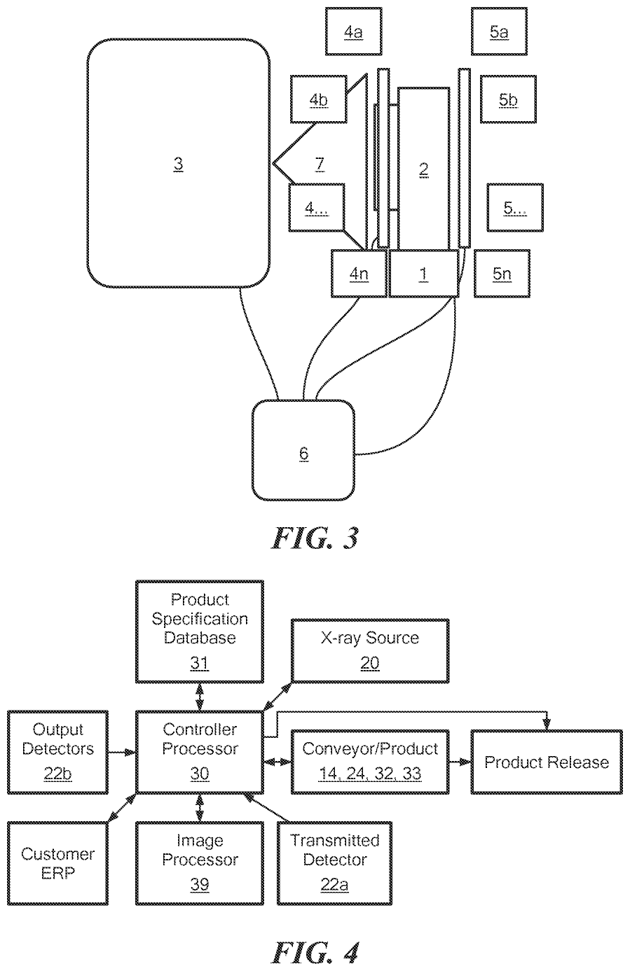

Controller subsystem 30, FIG. 3 receives the output of detection subsystems 22a and 22b and is configured (e.g., programmed) to determine an X-ray dose absorbed by the products. The controller subsystem may include one or more computers, controllers such as Applications Specific Integrated Circuits, microcontrollers, or the like.

In one example, the controller subsystem 30 is also configured to control the conveyance subsystem, for example conveyors 14 and 32 and return conveyor 33 which directs pallets from exit conveyor 32 back to entrance conveyor 14 and then to the treatment zone conveyor 24 to irradiate the pallet with an additional dose of X-rays in situations where the first measured dose was insufficient. As such, controller subsystem 30 may control pusher bars, gates, and the like used in conveyance systems. In one example, a typical pallet spends 15 to 30 seconds in the treatment zone and may return to the treatment zone 10 to 80 times for a sufficient dose. A proper sterilization may require 15 to 30 minutes in the treatment zone. Even so, the new process can provide for complete sterilization much faster than gamma radiation process. Accordingly, the controller subsystem may calculate the absorbed dose each time the pallet enters the treatment zone and return the pallets to the treatment zone until a specified dose (e.g., predetermined) is received.

The controller subsystem may also control the conveyor speed and/or the X-ray source (for example to increase or decrease the strength of the X-rays at the treatment zone). By controlling the conveyor speed, the controller subsystem can increase or decrease the dwell time that the products remain in the X-ray treatment zone. An approved dose for the medical devices being treated may be based on product specification stored in database 31, FIG. 3.

Other conveyance configurations are possible such as conveyances which rotate the pallet and/or hang the pallet from overhead rails, for example. And, the configuration shown in the figures are single level, but double level or alternative configurations can be used. Pallets of product are preferably loaded onto the entrance conveyor 14. The product enters the X-ray beam. An input detector 22a which may include one or more sensors/detectors measures the dose at the front of the product. The product absorbs and attenuates the X-rays flux. Detection subsystem 22b which could also include one or more sensors/detectors, measures the dose after it is transmitted through the product. The process control subsystem reads the measured dose and commands the X-ray source, the detectors (e.g., to measure baseline noise and for calibration), and the conveyor with an algorithm that evaluates when the product is properly sterilized. In the example described above, the control subsystem determines if the product needs to return to the treatment zone to receive more X-ray doses or instead leaves on the exit conveyor 32. The control subsystem preferably interacts with product specification database 31 which describes the products and which includes features such as quantity, density, minimum dose, maximum dose, and the like. The control subsystem preferably monitors and controls the X-ray source and the conveyor. The control subsystem reads out the output and reads out the signals from the detection subsystems and evaluates the data/information from these sources to decide when the product should be released.

In order to validate the sterilization process for the product, the prior art method required placing dosimeters by hand on the product. Once the product completed the sterilization process, the dosimeters had to be removed by hand and then measured to determine if the product could be released. This new system and method provides for real time measurement of the dose and the sterilization level for each product which can eliminate the need for dosimeters to be placed by hand and read out manually.

In one example, the products enter the sterilization vault, step 60, FIG. 4 and the treatment zone in the vault where the X-rays create a field of X-rays, step 62. Based on the output of the detectors, it can be determined whether or not the X-ray source is within specified tolerances, step 64, and if not, the source can be adjusted, step 66. The product is irradiated by X-rays for a dwell time, step 68, and based on the detector output, the input and output dose is measured, step 70. If sterilization on one side of the pallet is not complete, step 72, the product returns to the treatment zone as discussed above. If sterilization on one side of the product is complete, step 72, the pallet can be rotated, for example 180 degrees, step 74 (for example using conveyances which rotate the pallet) and the rotated product now enters the treatment zone, step 62.

One advantage of measuring the dose in real time is that if the X-ray source parameters, for example intensity, become out of compliance, the process can be stopped until the X-ray machine is brought back into compliance. This prevents product from completing the sterilization process and subsequently determining that the dose was not within the required specifications.

The inline dosimetry provides parametric release data that the sterilization process was controlled and that the process meets sterility requirements. Thus, the subject system and method can be used to comply with 21 CFR 211.165(a), and 211.167(a).5. Meeting the requirements of the parametric release process can provide greater assurance that a batch meets the sterility requirement that can be achieved without a sterility test of finished units drawn from the batch. As part of the parametric release program, the operational parameters that govern the delivery of the dosage would include the stacking configuration within the irradiation carrier or product, the bulk density of the product, the speed of the conveyor or carrier system, the distance to the radiation source and the duration of the product exposure. Demonstration of consistency in the absorbed radiation dosage at areas of minimum and maximum zones of radiation absorption within the fully loaded carriers on a batch to batch basis can provide for dosimetric release of radiation-sterilized medical and pharmaceutical products.

As an example of the algorithm/system operation for a pallet of low-density homogenous products, consider a pallet filled with boxes of nasal swabs with an overall density of 0.02_{g/cc}. As the product passes in front of the X-ray beam, attenuation of the X-ray beam occurs as it passes through the material via the equation:

I_{f}=Iₒe^{-μρl} (1)

Where μ is the attenuation coefficient, ρ is the material density, l is the path length, Iₒ is the measured X-ray flux entering the products and I_{f} is measured X-ray flux exiting the product. The X-ray field is preferably very high energy (e.g., 7,000,000 electron volts) and thus the average attenuation coefficient is very similar for most materials. Thus, the density of the products can be calculated by processor 30, FIG. 3.

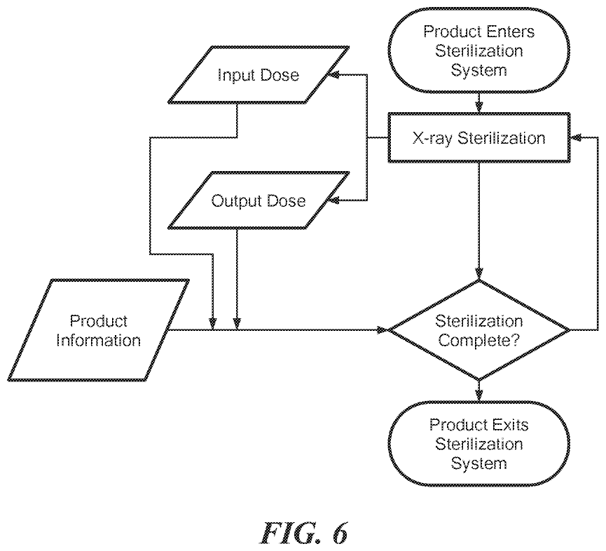

In another example, consider product with a density of 0.5 g/cc. For this product the total dose is shown in FIG. 8. If the D_min is 25 kGy the maximum dose at the edges would be 39 kGy. If product 2 has a D_max of 37 kGy then this product would not meet the specification. The system would be capable of detecting this situation early in the sterilization process and alerting the operator to the issue.

For several medical products, for example orthotic implants (e.g., hip, knee), the implant is made of metal and therefore has a localized, high-density volume. For a pallet of these products in certain scenarios there may be cases where one or more multiple implants shield another. In this scenario, the dose of the product will be a mix between the previous two examples. The system can measure and detect the doses, and may take into account the type, geometry, and density of the medical devices in order to assure the appropriate dose of radiation is absorbed by all the medical devices. For some situations, the real time dose measurements coupled with the map of the product can be used to ensure dose compliance. The system can adjust the dose on-the-fly for specific regions within the product to maintain dose compliance.

One or more detectors 22 can be used along with processor 39 to provide 2-D images of the products or even 3-D images of the products if computed tomography techniques are employed. In this way, along with knowing the density of the products, various actions can be taken by controller 30, FIG. 3.

For example, if density profile of the palletized products stored in database 31 does not match the calculated density, that could be an indication, for example, that one or more boxes were not packed the same as the other boxes. And, X-ray image information, for example, could be used to detect the overall configuration of the packaged products to ascertain, perhaps along with density information, if some high-density products are shielding less dense products. In response, the operator can be alerted that one or more boxes do not meet the stored (expected) product information.

Or, the X-ray dose can be automatically increased or decreased when density and/or image information reveals the products undergoing sterilization do not match (within some level of tolerance) the stored product/packing data. In but one example, if the stored product information indicates the products are low density products, but the calculated density indicates the products are high density products and result in high X-ray attenuation, then controller 30, FIG. 3 can automatically control the conveyor subsystem to increase the number of times the palletized products returns to the treatment zone.

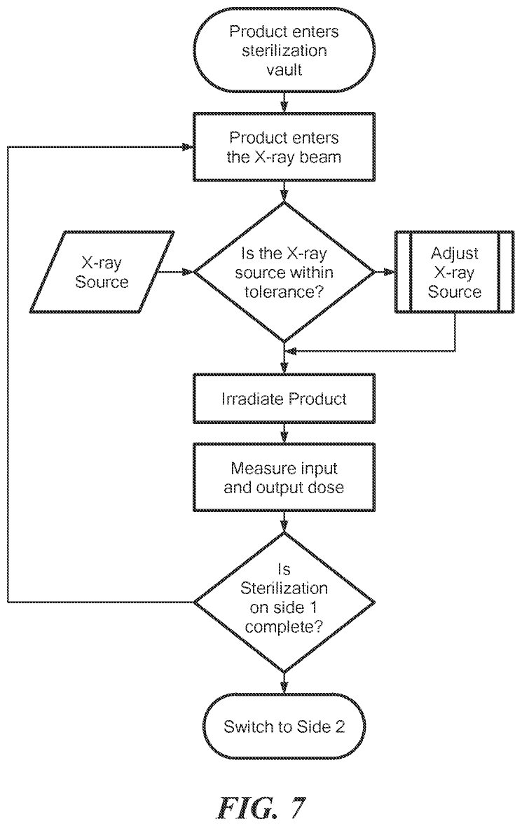

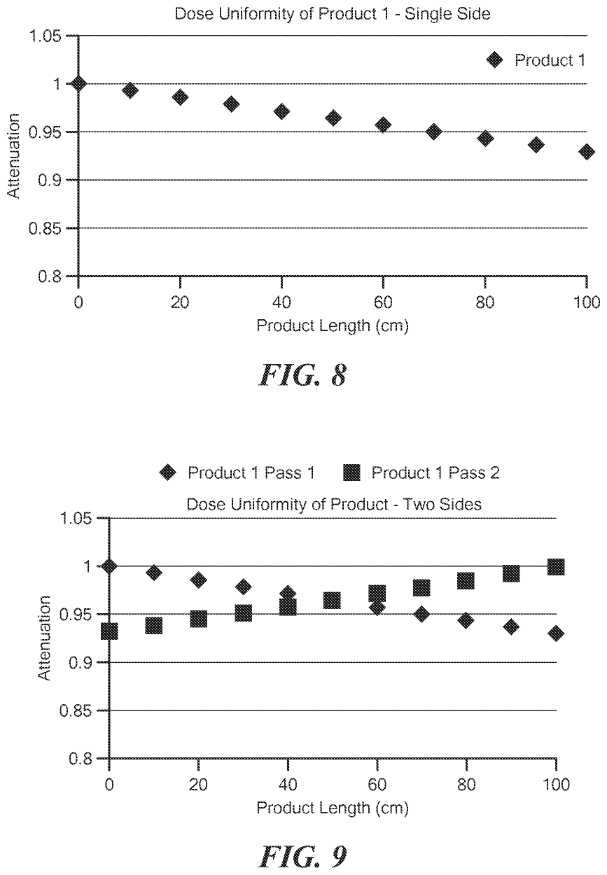

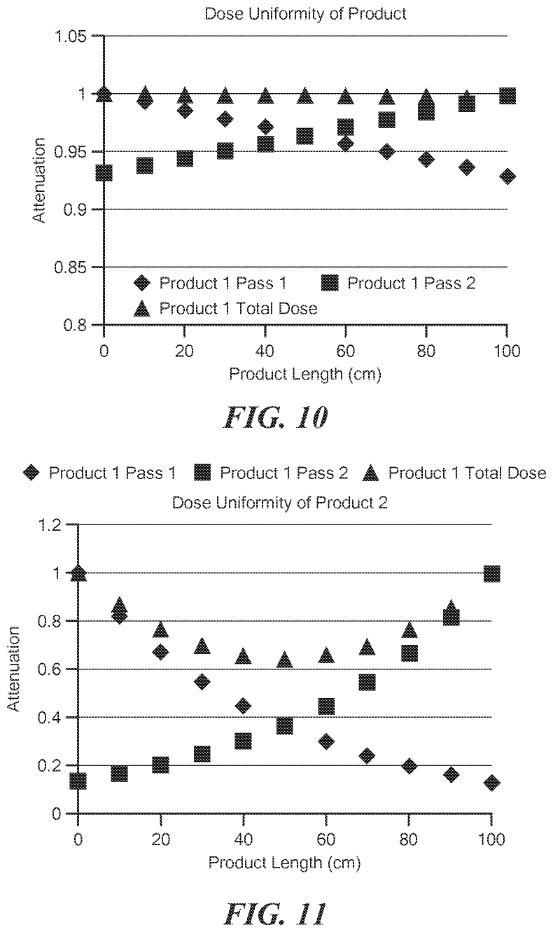

Most products have a dose minimum and maximum limit and the inline dose monitoring disclosed herein allows for a determination of whether or not the product meets these required doses. Incremental control is possible to achieve minimum and maximum dose requirements. See FIGS. 8-11. The method and system allow for feedback to make sure the product has received the required dose, determine unique density profiles, or adjust the product speed in the radiation field. The system can provide for a profiled X-ray intensity or dose for specific products or to make sure any product gets the appropriate dose. The system can be used to ensure uniformity of the dose to the product. The system and method can enable tracking the performance of the X-ray source to determine and schedule maintenance and/or repair. Furthermore, the irradiation duration and sequence can be specified for a specific product to meet compliance and be subsequently measured and confirmed with this system. As such use of this system in the irradiation process enables sterilization of the product to compliance specifications in a different facility with minimal need for recertification of the process from a compliance point of view. The system can be scaled and applied to the same product in a different facility that may be operating with a higher or lower power X-ray source. This presents unique advantages in rapid sterilization of products due to transportation disruptions due to weather or any other events by sterilizing the product at a geographically accessible facility while maintaining dose compliance. The system and method can enable calibration of the dose sensors to check or confirm with standard dosimetry practices. The method preferably allows for computing the absorbed dose to the product. The method and system can utilize real time adjustments of dose profile based on measured attenuation/density profiles for products with non-uniform density or packaging. An image of the irradiated product can be developed and that image can be compared against library data to confirm compliance. This can ensure, for example, that what is stated to be packaged on a pallet actually matches the products packaged on the pallet. Non-compliant product can be flagged based on real time measurement of the transmitted dose through the system. The method also enables a higher level of dose compliance regardless of product movement during shipment.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A product sterilization system comprising:
a vault;
a source of X-rays in the vault producing a field of X-rays in a treatment zone;
a conveyance delivering products to the treatment zone for irradiation by the field of X-rays;
a first X-ray detection subsystem in the treatment zone between the products and the source of X-rays;
a second X-ray detection subsystem in the treatment zone behind the products; and
a controller subsystem responsive to the first X-ray detection subsystem and the second X-ray detection subsystem and configured to determine an X-ray dose absorbed by the products.

2. The system of claim 1 in which the source of X-rays includes an electron accelerator and a target producing X-rays in response to electrons emitted by the electron accelerator.

3. The system of claim 1 in which the conveyance includes a vault entrance conveyor, a vault exit conveyor, and a return conveyor between the entrance conveyor and the exit conveyor configured to return products to the treatment zone.

4. The system of claim 1 in which the controller subsystem is further configured to direct the products from the exit conveyor to the return conveyor when the dose absorbed by the products is below a specified dose.

5. The system of claim 1 in which the first X-ray detection subsystem includes one or more low attenuation detectors between the product and the source of X-rays.

6. The system of claim 5 in which said detectors are ion chamber detectors.

7. The system of claim 1 in which the products are palletized.

8. A product sterilization method comprising:
conveying products to a treatment zone irradiated by a field of X-rays;
measuring the intensity of the X-rays in the treatment zone before the X-rays irradiate the products;
measuring the intensity of the X-rays in the treatment zone behind the products after the X-rays pass through the products; and
automatically determining an X-ray dose absorbed by the products based on the measured intensity of the X-rays in the treatment zone before the X-rays irradiate the product and the measured intensity of the X-rays in the treatment zone behind the products after the X-rays pass through the products.

9. The method of claim 8 further including, automatically calculating when the dose absorbed by the products is below a specified dose and if so automatically returning the products via a conveyance to the treatment zone.

10. A product sterilization method comprising:
conveying products to a treatment zone irradiated by a field of X-rays;
where the X-ray flux before and after traversing through the products is mapped, calibrated and stored in a database; and
with such information either directly or scaled applied to the same process in a different facility with an X-ray source of either the same or different power.
